(19) United States
(12) Patent Application Publication
(10) Pub. No.: US 2013/0116282 A1
(43) Pub. Date: May 9, 2013

(54) BUCKWHEAT EXTRACT ENRICHED IN D-FAGOMINE

(71) Applicants: Josep Lluis Torres Simón, Barcelona (ES); Susana Amézqueta Pérez, Martorell (ES); Sergio Pumarola Segura, Barcelona (ES); Pere Clapés Saborit, Vilassar De Dalt (ES)

(72) Inventors: Josep Lluis Torres Simón, Barcelona (ES); Susana Amézqueta Pérez, Martorell (ES); Sergio Pumarola Segura, Barcelona (ES); Pere Clapés Saborit, Vilassar De Dalt (ES)

(73) Assignee: BIOGLANE, S.L.N.E., Barcelona (ES)

(21) Appl. No.: 13/808,378

(22) PCT Filed: Jul. 15, 2011
(86) PCT No.: PCT/EP11/62143

§ 371 Date: Jan. 4, 2013

(30) Foreign Application Priority Data
Jul. 16, 2010 (EP) 10382199.7

(51) Int. Cl. A61K 31/445; A23L 1/30

(52) U.S. Cl. CPC A61K 31/445; A23L 1/3002
USPC 514/315; 426/61; 426/2

(57) ABSTRACT

The buckwheat extract comprises a extract of buckwheat which are enriched in D-fagomine and including 3,4-di-epifagomine, a process for their preparation, pharmaceutical or veterinary compositions, dietary supplements or functional foods containing them, their use alone or in combination with saccharide, an iminocyclitol or probiotics, as a blood glucose levels controlling agent reducing the post-prandial glucose levels, as well as the buckwheat extract for use in the prevention and/or coadjuvant treatment of microbiota imbalance, that reduces the adhesion of some potentially harmful microorganism in the microbiota and therefore increasing the resistance to disease.

The present invention relates to extracts of buckwheat which are enriched in D-fagomine, a process for their preparation, pharmaceutical or veterinary compositions, dietary supplements or functional food containing them, and their use as a blood glucose levels controlling agent reducing the post-prandial glucose levels, as well as their use for the prevention and/or coadjuvant treatment of microbiota imbalance, reducing the adhesion of some potentially harmful microorganism in the microbiota and increasing the resistance to diseases.

### BACKGROUND ART

D-fagomine is the International Non-proprietary Name of (2R,3R,4R)-2-(hydroxymethyl)piperidine-3,4-diol. D-fagomine is an iminocyclitol isolated from buckwheat seeds in 1971 and its structure was elucidated in 1975 corresponding to formula (I):

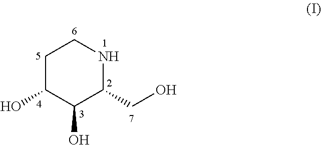

D-fagomine is known as an alpha-glycosidase and lactase weak inhibitor (Cf. Atsushi Kato et al., Fagomine isomers and glycosides from Xanthocercis zambesiaca. Journal of Natural Products 1997, vol. 60, pp. 312-314) with antihyperglycemic effect (Cf. Hiroshi Nojima et al., “Antihyperglycemic effects of N-containing sugars from Xanthocercis zambesiaca, Morus bombycis, Aglaonema treubii, and Castanospermum australe in Streptozotocin-diabetic mice” Journal of Natural Products, 1998, vol. 61. pp. 397-400). Natural sources of D-fagomine are different species of plants, such as Morus Bombycis or Fagopyrum esculentum (buckwheat) (Cf. Asano N. et al., Sugars with nitrogen in the ring isolated from the leaves of Morus bombycis, Carbohydrates Research 1994, vol. 254, pp 235-245 and Iqbal et al., Allelopathy of buckwheat: Assessment of allelopathic potential of extract of aerial parts of buckwheat and identification of fagomine and other related alkaloids as allelochemicals, Weed Biology and Management, 2002, vol. 2, pp. 110-115).

3,4-di-epifagomine is the International Non-proprietary Name of (2R,3S,4S)-2-(hydroxymethyl)piperidine-3,4-diol. It is the isomer (2R,3S,4S)-form of D-fagomine and its structure corresponding to formula (II).

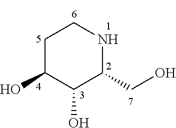

The natural source is Xanthorcesis zambesiaca (Cf. Kato et al., Fagomine isomers and glycosides from Xanthocersis zambesiaca, 1997, vol. 60, pp. 312-314).

Many positive physiological effects are associated with buckwheat, due to the presence of soluble and insoluble fibers, antioxidant substances as well as the absence of glutenin-like proteins. While soluble and insoluble dietary fibers have a positive effect on constipation and obesity, antioxidants may play an important role in lipid antioxidation and cancer prevention.

Extracts are concentrated preparations of various parts of plants obtained by isolating the active constituents, such as D-fagomine, from the plant by suitable means. Depending of the physico-chemical features of these active ingredients, suitable means for their isolation can be used, for example, aqueous solutions, organic solvents, microwave or supercritical fluids extraction.

Plant extracts contain not only one but multiple constituents, many of them biologically active. Often, the beneficial effect is derived from the combination of many of these active compounds, even though in some cases there is one particular compound that is mainly responsible for most of the activity. United States patent application US 20080014294 discloses the use of buckwheat extracts containing components for managing serum glucose levels in humans, and a method for the extraction of these components from buckwheat seeds by sequentially extraction steps with non-polar, polar protic and/or polar aprotic solvents.

Recent studies have shown the increasing interest in the use of buckwheat as a brewing ingredient for the production of gluten-free beer. These beers are produced by an alcoholic fermentation of sugary wort, using a yeast of Saccharomyces genus, such as Saccharomyces carlsbergensis (Cf. European patent application EP 949328; and Blaise P. Nic Phiarais, “Use of response surface methodology to investigate the effectiveness of commercial enzymes on buckwheat malt for brewing purposes”, Journal of the Institute of Brewing, 2006, vol. 114(4), pp. 324-332).

Active ingredients extracted from plants are sometimes directly incorporated into food or beverages, as well as into pharmaceutical or cosmetic compositions in a variety of forms, including a pure or semi-pure component, a solid or liquid extract, or a solid plant matter. In particular, United States patent application US20010018090 discloses a calorie reduced food or beverage which contains 1-deoxynojirimycin, or some of their analogues, including D-fagomine.

Buckwheat extracts may contain other components such as inositols including D-chiroinositols (e.g. fagopyritols) and mio-inositols; polyphenols including flavonoids; fermentable or non-fermentable sugars; and proteins.

Buckwheat seeds are substantially free of 1-deoxynojirimycin (DNJ) and of 1,4 dideoxy-1,4-imino-D arabinitol (D-AB1), otherwise the mulberry roots and leaves contain 1-deoxynojirimycin and 1,4 dideoxy-1,4-imino-D arabinitol, which have been described by Watson et al. “Polyhydroxylated alkaloids natural occurrence and therapeutic applications”, Phytochemistry, 2001, vol. 56, pp. 265-295.

High amounts of buckwheat extract would have to be used in order to obtain a high amount of D-fagomine.

Although several processes for the extraction of D-fagomine have already been described, there is still a need of a simple and economic process which allows obtaining at industrial scale of extracts of buckwheat extracts enriched in D-fagomine, almost substantially free from components with other activities, with no remarkable losses or alterations in the active ingredient.

### SUMMARY OF THE INVENTION

The inventors have found a process for preparing extracts of buckwheat with a high content of D-fagomine and including 3,4-di-epifagomine, which comprises an alcoholic fermentation, as well as purification steps based on the use of adsorption resins, and ion-exchange resins, these steps being carried out in an appropriate order. This specific combination of steps achieves the removal of undesirable and/or inactive compounds from the natural extract and increases the concentration of D-fagomine in the extract.

Accordingly, a first aspect of the present invention refers to a buckwheat extract which comprises an amount of D-fagomine comprised between 2% and 40% by weight of dry mass, and 3,4-di-epifagomine, wherein the weight ratio of 3,4-di-epifagomine/D-fagomine is comprised between 1:10 and 1:1 and the extract is substantially free of 1-deoxynojirimycin and 1,4 dideoxy-1,4-imino-D-arabinitol.

A second aspect of the present invention refers to a process for the preparation of the extract as defined above, which comprises: (a) milling the buckwheat, passing a sieve, and mixing it with water; (b) mashing the mixture of step (a); (c) carrying out an ethanolic fermentation of the extract obtained in step (b); (d) passing the fermented extract obtained in step (c) through a cation exchange resin, whereby the D-fagomine is retained; (e) eluting the retained D-fagomine from the resin of step (d) with an alkaline buffer; and (f) passing the extract obtained in step (e) through an anion exchange resin whereby D-fagomine is eluted directly.

A third aspect of the present invention refers to a functional food, dietary supplement, pharmaceutical or veterinary composition, which comprises the extract of the present invention.

A fourth aspect of the present invention refers to the non-therapeutic use of the extract as defined in the first aspect as a blood glucose levels controlling agent to reduce post-prandial blood glucose levels after carbohydrate intake.

A fifth aspect of the present invention refers to the extract of the present invention for the prevention and/or coadjuvant treatment of a microbiota imbalance caused by enteric or oral bacteria. The extract reduces the adhesion of some potentially harmful microorganism in the microbiota therefore increasing resistance to diseases.

Finally, a sixth aspect of the present invention refers to a process for the preparation of a substantially pure D-fagomine comprising carrying out the process as defined in the second aspect of the invention further comprising an additional step of passing the eluted fraction from step (f) through a high resolution cation exchange resin with terminal carboxymethyl groups whereby the D-fagomine is retained, and eluting the retained D-fagomine with an alkaline buffer.

### BRIEF DESCRIPTION OF THE DRAWINGS

FIG. 1A shows the MALDI-TOF (Matrix-Assisted Laser Desorption/Ionization-Time-Of-Flight) analysis results of the non-retained materials in cation exchange resin of Example 1.

FIG. 1B shows the MALDI-TOF (Matrix-Assisted Laser Desorption/Ionization-Time-Of-Flight) analysis results of the retained materials in cation exchange resin of Example 1.

FIG. 2A shows the HPLC-UV profile at 214 nm of a boiled wort sample from Example 2.

FIG. 2B shows the HPLC-UV profile at 214 nm of a sample after the purification by adsorption resin (B) of Example 2.

FIG. 2C shows the HPLC-UV profile at 214 nm of a sample after the fermentation step (C) of Example 2.

FIG. 3A shows the MALDI-TOF analysis results of the non-retained materials in cation exchange resin of Example 2.

FIG. 3B shows the MALDI-TOF analysis results of the retained materials in cation exchange resin of Example 2.

FIG. 4 shows the structure elucidation by RMN of the compound at m/z 148.2, t_{R}=25 min, determined by HPLC-MS in Example 4. H₁-H₇ refers to the position of the H on the carbons of the structure. The numbers on the carbons are those indicated on the structure of D-fagomine (cf. formula D-fagomine in the background art section)

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

The term “substantially free of fermentable sugars” as used herein refers to an extract that contains 10% by weight or less of fermentable sugars. These percentages are based upon the total amount of fermentable sugars presents in the extract. Preferably, the extract contains 5% by weight or less of fermentable sugars. These percentages are based upon the total dry extract mass.

The term “substantially free of 1-deoxynojirimycin” as used herein refers to an extract that contains 1% by weight or less of 1-deoxynojirimycin. These percentages are based upon the total dry extract mass.

The term “substantially free of 1,4 dideoxy-1,4-imino-D arabinitol” as used herein refers to an extract that contains 1% by weight or less of 1,4 dideoxy-1,4-imino-D arabinitol. These percentages are based upon the total dry extract mass.

The term “fermentable sugars” as used herein refers to sugars that can be converted to alcohol and CO₂. Examples of fermentable sugars include glucose, xylose, maltose, arabinose, fructose or sucrose.

The term “mashing” as used herein refers to the process of heating a mixture of milled seed or grain and water, allowing the enzymes present in the malt to break down the starch in the grain or seed into fermentable sugars. The liquid thus obtained is called wort. This wort contains the suitable sugars to be fermented in order to produce alcohol.

The term “adsorbent resin” as used herein refers to porous spherical polymers which their high internal surface areas can adsorb and then desorb a wide variety of substances. These substances are trapped and removed from the flow of the mobile phase depending on their effective size and polarity. Examples of adsorbent resin include crosslinked, macroreticular polystyrene, and aliphatic polymer.

The term “ion exchange resin” as used herein refers to a type of resin that attaches ions onto it. Solute ions in the mobile phase of the opposite charge to the stationary phase are attracted to the resin by electrostatic forces.

The term “cation exchange resin” as used herein refers to a type of ion exchange resin which retains positively charged ions, due to the fact that the stationary phase displays a negatively charged functional group.

The term “anion exchange resin” as used herein refers to a type of ion exchange resin which retains negatively charged ions, due to the fact that the stationary phase displays a positively charged functional group.

The term “glucemic index” or “glycemic index” as used herein interchangeably refers to the area under the two hour blood glucose response curve (AUC) following the ingestion of a fixed portion of carbohydrate, usually 50 g. The term “glycemic load” refers to glycemic index multiplied by the carbohydrate intake. “Glycemic load” may also be related to the total glucose absorbed over 2 hours from ingestion.

The term “fagopyritol” as used herein refers to an unspecified alpha-galactosyl-D-chiro-inositol, its salts or its derivatives.

The term “dietary supplement”, “food supplement” or “nutritional supplement” as used herein interchangeably refers to a preparation intended to supplement the diet and provide nutrients, such as vitamins, minerals, fiber, fatty acids, or amino acids, that may be missing or may not be consumed in sufficient quantity in a person's diet.

The term “enteric bacteria” as used herein refers to a microorganism that lives, resides, occupies or populates the intestines.

The term “oral bacteria” as used herein refers to microorganism that lives, resides, occupies or populates teeth surface and gingival epithelium.

The term “saccharide” or “carbohydrate” as used herein interchangeably refers to an organic compound which consists only of carbon, hydrogen and oxygen, with the last two in the 2:1 atom ratio, which may be a source of energy or an analogue of epithelial cells surface polymers.

The term “probiotic” as used herein refers to a live microorganisms which, when administered in adequate amounts, confer a health benefit on the host.

The term “iminocyclitol” as used herein refers to any amino derivative of a cyclitol, being a cyclitol any hydroxylated cycloalkane having at least three hydroxy groups attached to different carbon atoms.

The term “functional food” as used herein refers to any healthy or functional food which helps to maintain the body functions beyond the basic role of supplying nutrients.

The term “functional beverage” refers to drinks that have been enhanced with added ingredients which help to maintain the body functions beyond basic nutrition.

The term “alkaline buffer” or “alkaline buffer solution” as used herein interchangeably refers to an aqueous solution consisting of a mixture of a weak base and its conjugate acid which provides an alkaline pH. The buffer solutions keep the pH at a nearly constant value. Suitable alkaline buffer includes ammonia solution, potassium carbonate, sodium carbonate, potassium bicarbonate, sodium bicarbonate, sodium hydroxide, potassium hydroxide, calcium hydroxide, diammonium phosphate, sodium phosphate, ammonium acetate, sodium citrate, tris (hydroxymethyl) aminomethane or sodium benzoate.

The term “weight controlling agent” as used herein refers to an agent able to maintain a constant weight, to reduce weight gain or to lose weight.

The term “% by weight of the dry extract mass” as used herein refers to a mass percent or weight percent (w/w), for example an amount of D-fagomine of 2% by weight of dry extract mass refers to 2 units of D-fagomine in 100 units of dry extract mass.

As mentioned above, the first aspect of the present invention refers to a buckwheat extract comprising an amount of D-fagomine comprised between 2% and 40% by weight of dry extract mass and 3,4-di-epifagomine, wherein the weight ratio of 3,4-di-epifagomine/D-fagomine is comprised between 1:10 and 1:1, and the extract is substantially free of 1-deoxynojirimycin and 1,4 dideoxy-1,4-imino-D-arabinitol. The increase in the amount of D-fagomine and the presence of 3,4-di-epifagomine in the buckwheat extracts as mentioned above is accompanied by a reduction in other active components of the extract. This is advantageous because the use of a lower amount of the extract is needed to achieve the active concentration for the inhibitory activity of the alpha-glucosidase in the small intestine. Therefore, the release of monosacharides from disaccharides (sugars) or polysaccharides (starches) is slowed down.

The weight ratio of 3,4-di-epifagomine/D-fagomine in the buckwheat plant can vary within 1:1-1:10 depending on several conditions such as variety, as it is shown in Example 8 Table 4. In a preferred embodiment, the weight ratio of 3,4-di-epifagomine/D-fagomine is comprised between 1:4 and 1:1. In a more preferred embodiment, the weight ratio of 3,4-di-epifagomine/D-fagomine is 1:2.

In a more preferred embodiment, the buckwheat extract of the present invention comprises an amount of D-fagomine comprised between 5% and 18% by weight of dry extract mass. In an even more preferred embodiment, the buckwheat extract of the present invention comprises an amount of D-fagomine comprised between 9% and 18% by weight of dry extract mass. In a still even more preferred embodiment, the buckwheat extract of the present invention comprises an amount of D-fagomine comprised between 12% and 18% by weight of dry extract mass. In a particular embodiment, the amount of D-fagomine is 18% by weight of dry extract mass.

In a preferred embodiment, the buckwheat extract as mentioned above comprises an amount of D-fagomine by weight of dry extract mass selected from the following amounts: 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% and 40%.

Due to the structural similarity between 3,4 Di-epifagomine and D-fagomine, 3,4 Di-epifagomine may also act as an alpha-glucosidase inhibitor.

In another preferred embodiment, the buckwheat extract of the present invention is substantially free of fermentable sugars. Examples of these fermentable sugars include, but are not limited to, glucose, xylose, maltose, arabinose, or fructose.

In a particular embodiment, the buckwheat extract of the present invention contains a percentage of fermentable sugars equal or lower to 10% of fermentable sugars. In another particular embodiment, the buckwheat extract of the present invention contains a percentage of fermentable sugars equal or lower to 8% of fermentable sugars. In an even another particular embodiment, the buckwheat extract of the present invention contains a percentage of fermentable sugars equal or lower to 6% of fermentable sugars. Preferably, the buckwheat extract of the present invention contains a percentage of fermentable sugars equal or lower to 5% of fermentable sugars.

As mentioned above, the enriched buckwheat extract of the present invention can be prepared by a process comprising the following steps: (a) milling the buckwheat, passing a sieve, and mixing it with water; (b) mashing the mixture of step (a); (c) carrying out an ethanolic fermentation of the extract obtained in step (b); (d) passing the fermented extract obtained in step (c) through a cation exchange resin whereby the D-fagomine is retained; (e) eluting the retained D-fagomine from the resin of step (d) with an alkaline buffer; and (f) passing the extract obtained in step (e) through an anion exchange resin whereby the D-fagomine is eluted directly.

The temperature reached during the mashing step allows the reduction of sugar content. The mashing of step (b) comprises the addition of external enzymes or malted cereals milled to carry out the hydrolysis of complex sugars into fermentable sugars.

In a preferred embodiment, the mashing step (b) is carried out by heating the aqueous extract obtained in step (a) in the presence of exogenous enzymes as it is shown in Examples 1, 3, and 4. In a particular embodiment, the exogenous enzymes are selected from the group consisting of alpha-amylase, beta-amylases, protease, beta-gluconase, pululanase, amiloglucosidase, and their mixtures.

In another preferred embodiment, the mashing step (b) is carried out by heating the aqueous extract obtained in step (a) in the presence of malted cereals as it is shown in Example 2. In a particular embodiment, malted cereals which can be added are selected from the group consisting of buckwheat, rice, corn, sorghum, millet, barley and their mixtures.

As it is shown in the Examples, the mashing step (b) of the process of the present invention which is carried out either by the addition of endogenous enzymes (cf. Example 1) or by the addition of malted cereals (cf. Example 2) allows the appropriate starch breakdown and reduction of sugar content to carry out the fermentation step (c). In both cases, the extract mass obtained after the fermentation step (c) is reduced up to 40% after sugar consumption. This fact contributes to achieve the claimed content of D-fagomine in dry extract mass of the present invention.

Preferably, the mashing step (b) is carried out by heating the aqueous suspension of buckwheat at a temperature range comprised between 30° C. and 100° C. More preferably, the temperature of the mashing step is comprised between 40° C. and 80° C.

In a preferred embodiment, the process for preparing the buckwheat extract of the present invention further comprising an additional step of boiling the extract obtained in step (b), and clarifying the wort obtained by centrifugation or decantation. The sediment fraction is then collected.

The addition of the boiling step in the process of the present invention does not modify the claimed amount of D-fagomine in the extract of the present invention. Nevertheless, the boiling step allows the reduction of the protein content of the intermediate worts making easier the manipulation of these intermediate worts. Additionally, as it is widely known in the beer industry, the addition of a boiling step also allows the pathogen removal.

Optionally, the extract obtained in step (b) can be subjected to a purification process before the fermentation step, by passing the extract obtained in step (b) or after the additional step of boiling through an adsorption resin whereby the D-fagomine is eluted directly. The addition of the adsorption resin allows the removal of proteins and polyphenols from the extract. Proteins are usually of high molecular weight, and polyphenols are mixtures of substances with a huge range of molecular weight and polarity. Examples of adsorption resins include, but are not limited to, macroreticular non-functionalised resin or gel filtration eluted by gravity. In this preferred embodiment, the extract obtained through the adsorption resin is then subjected to a fermentation step. In preferred embodiment, the volume of the solution of the extract obtained after the elution through the adsorption resin is reduced up to the same volume of the initial solution of the extract. It has been found that under these working conditions the fermentability of the extract is higher, allowing a high removal of the fermentable sugar present in the extract.

An ethanolic fermentation is a biological process in which fermentable sugars such as glucose, fructose, and sucrose are converted into cellular energy, producing ethanol, and carbon dioxide as metabolic waste products. The most commonly used yeast for performing this fermentation is Saccharomyces genus yeast. Species of Saccharomyces genus yeast suitable for the present invention are selected from the group consisting of Saccharomyces cerevisiae, Saccharomyces pombe, Saccharomyces carlsbergensis, and their mixtures. Preferably, the specie of Saccharomyces is Saccharomyces cerevisiae.

In a particular embodiment, the ethanolic fermentation is carried out at a temperature comprised between 10° C. and 25° C. In another particular embodiment, the temperature range of the ethanolic fermentation is comprised between 10° C. and 20° C. In another particular embodiment, the temperature range of the ethanolic fermentation is comprised between 13° C. and 16° C. Preferably, the temperature of the ethanolic fermentation is 14° C.

The fermented extract obtained in step (c) is then subjected to a second purification process, by passing the extract through a cation exchange resin. The D-fagomine is retained in the resin and then eluted with an alkaline buffer. Appropriate alkaline buffer for eluting the retained D-fagomine are selected from the group consisting of ammonia solution, potassium carbonate, sodium carbonate, potassium bicarbonate, sodium bicarbonate, sodium hydroxide, potassium hydroxide, calcium hydroxide, diammonium phosphate, sodium phosphate, ammonium acetate, sodium citrate, tris (hydroxymethyl) aminomethane or sodium benzoate. In a preferred embodiment the alkaline buffer is the ammonia solution

Cation exchange resins include weak or strong acid resins. Weak acid resins are functionalized by carboxylic acid groups, and strong acid resins are functionalized by sulfonic groups. In a particular embodiment, the extract obtained in step (c) is passed through a cation exchange resin being the resin a weak acid exchange resin. In another particular embodiment, the extract obtained in step (c) is passed through a cation exchange resin being the resin a strong acid exchange resin.

The purified extract obtained in step (e) is subjected to a third purification process, by eluting the extract through an anion exchange resin, whereby the D-fagomine elutes directly. Anion exchange resins include weak or strong basic resins. Weak basic resins are functionalized by primary, secondary, and/or ternary amino groups, such as polyethylene amine, and strong basic resins are functionalized by quaternary amino groups, for example, trimethylammonium groups. These resins allow the removal of anions.

In a particular embodiment, the extract obtained in step (e) is eluted through an anion exchange resin being the resin a strong basic anion exchange resin.

In a particular embodiment, the extract obtained in step (e) is eluted through an anion exchange resin being the resin a weak basic anion exchange resin.

In a particular embodiment, the extract obtained in step (f) is dried. The evaporation can be carried out by conventional methods including the use of a rotary evaporator, a spray dryer, a freeze dryer or any other conventional dryer.

In addition, the buckwheat extract obtainable by the above mentioned process which comprises: (a) milling the buckwheat, passing a sieve, and mixing it with water; (b) mashing the mixture of step (a); (c) carrying out an ethanolic fermentation of the extract obtained in step (b); (d) passing the fermented extract obtained in step (c) through a cation exchange resin, whereby the D-fagomine is retained; (e) eluting the retained D-fagomine from the resin of step (d) with an alkaline buffer; and (f) passing the extract obtained in step (e) through an anion exchange resin whereby D-fagomine is eluted directly.

In a preferred embodiment, it is provided a buckwheat extract obtainable by the above mentioned process which comprises (a) milling the buckwheat, passing a sieve, and mixing it with water; (b) mashing the mixture of step (a); (c) carrying out an ethanolic fermentation of the extract obtained in step (b); (d) passing the fermented extract obtained in step (c) through a cation exchange resin, whereby the D-fagomine is retained; (e) eluting the retained D-fagomine from the resin of step (d) with an alkaline buffer; and (f) passing the extract obtained in step (e) through an anion exchange resin whereby D-fagomine is eluted directly, the process further comprising an additional step of boiling the extract obtained in step (b), and clarifying the wort obtained by centrifugation or decantation.

In another preferred embodiment, it is provided a buckwheat extract obtainable by the above mentioned process which comprises (a) milling the buckwheat, passing a sieve, and mixing it with water; (b) mashing the mixture of step (a); (c) carrying out an ethanolic fermentation of the extract obtained in step (b); (d) passing the fermented extract obtained in step (c) through a cation exchange resin, whereby the D-fagomine is retained; (e) eluting the retained D-fagomine from the resin of step (d) with an alkaline buffer; and (f) passing the extract obtained in step (e) through an anion exchange resin whereby D-fagomine is eluted directly, or, alternatively, the previous process further comprising an additional step of boiling the extract obtained in step (b), and clarifying the wort obtained by centrifugation or decantation, both process further comprising passing the extract obtained in step (b) or after the additional step of boiling through an adsorption resin whereby the D-fagomine is eluted directly.

In another preferred embodiment, it is provided a buckwheat extract obtainable by the above mentioned processes further comprising drying the extract obtainable in step (f).

Another aspect of the present invention is a process for the preparation of a substantially pure D-fagomine. Thus, the process for the preparation of a substantially pure D-fagomine comprises: (a) milling the buckwheat, passing a sieve, and mixing it with water; (b) mashing the mixture of step (a); (c) carrying out an ethanolic fermentation of the extract obtained in step (b); (d) passing the fermented extract obtained in step (c) through a cation exchange resin, whereby the D-fagomine is retained; (e) eluting the retained D-fagomine from the resin of step (d) with an alkaline buffer; (f) passing the extract obtained in step (e) through an anion exchange resin whereby D-fagomine is eluted directly, the process further comprising an additional step of passing the eluted fraction from step (f) through a high resolution cation exchange resin with terminal carboxymethyl groups whereby the D-fagomine is retained, and eluting the retained D-fagomine with an alkaline buffer.

The term “substantially pure D-fagomine” as used herein refers that the D-fagomine contains 15% by weight or less of the presence of another compounds and particularly contains 15% by weight or less of 3,4 Di-epifagomine.

In a preferred embodiment the obtained substantially pure D-fagomine has a D-fagomine content of at least 90% by weight. In a more preferred embodiment the obtained substantially pure D-fagomine has a D-fagomine content of at least 95% by weight. In even more preferred embodiment the obtained substantially pure D-fagomine has a D-fagomine content of at least 98% by weight. Preferably, the obtained substantially pure D-fagomine has a D-fagomine content of at least 99% by weight.

The high resolution cation exchange resin with terminal carboxymethyl groups allows the removal of the stereoisomers of D-fagomine from the buckwheat extracts of the present invention. Each stereoisomers of D-fagomine can be eluted in different fractions. (cf. Castillo et al. “Supporting information:Fructose-6-phosphate aldolase in organic synthesis: preparation of D-fagomine, N-alkylated derivatives and preliminary biological assays”, Internet citation, 29 Nov. 2006, pp. S1-S29, ISSN: 1523-7060; Chen et al “Thermospray liquid chromatographic mass spectrometric analysis of castanospermine related alkaloids in castanospermun Australe”, Journal of Natural Products, 1990, vol. 53, n° 2, pp. 369-365; and “Practical methods for biocatalysis and biotransformation”, John Whittall et all, Ed. Wiley, page 216)

Appropriate high resolution cation exchange resin with terminal carboxymethyl groups for the present invention are selected from the group consisting of. Examples of commercial high resolution cation exchange resin are: CM Sepharose fast flow resin from GE Healthcare (Buckinghamshire, England) or CM Toyopearl 650 S from Tosoh Bioscience (Tokyo, Japan).

The extracts of the present invention can be in the form of a functional food or a, dietary supplement. It can also be in the form of a pharmaceutical or veterinary composition.

In a preferred embodiment, the extracts of the present invention are forming part of a functional food. They can be used as a food or a beverage additive to produce a functional food or a functional beverage. Thus, they can be added to semisolid products, solid products, or liquid products, or their derivatives such as concentrates or powders. Examples of food products are selected from the list consisting of milk and derivatives such as yoghurts or cheese; beverages including juices, soft drinks, sport drinks, or alcoholic beverages; confectionary such as chocolates, candies, or jellies; pasta; cereals; and bakery.

In a particular embodiment, the functional food is selected from the group consisting of beer, non-alcohol beer, tea, milk, pasta, biscuits, cookies, cereal bars, breakfast cereals, swollen grains, bread, crepes, pancakes, cakes, creams, and desserts. In a more preferred embodiment, the functional food is useful for infant administration, preferably as a part of an infant formula.

In another preferred embodiment the extracts of the present invention are in form of a dietary supplement. The dietary supplement comprises an effective amount of the extract as defined above together with one or more appropriate edible acceptable excipients or carriers.

In a particular embodiment the extracts of the present invention are in form of a pharmaceutical or veterinary composition. The pharmaceutical composition comprises an effective amount of the extract as defined above together with one or more appropriate pharmaceutically acceptable excipients or carriers. The veterinary composition comprises an effective amount of the extract as defined above together with one or more appropriate veterinary acceptable excipients or carriers. The excipients or carriers employed are for oral or vaginal administration, including but not limited to, fillers, binders, disintegrates, lubricants, anticaking, glidants or their mixtures.

The pharmaceutically or veterinary compositions, and dietary supplements of the invention can be formulated in several forms that include, but are not limited to, solutions, tablets, capsules, granules, suspensions, dispersions, powders, lozenge, chewable candy, candy bar, concentrate, drops, elixir, emulsion, film, gel, granule, chewing gum, jelly, oil, paste, pastille, pellet, soap, sponge, suppository, syrup, chewable gelatin form, or chewable tablet.

The compositions of the present invention can be prepared according to methods well known in the state of the art. The appropriate excipients and/or carriers, and their amounts, can readily be determined by those skilled in the art according to the type of formulation being prepared.

It has been reported that D-fagomine is an alpha-glycosidase inhibitor with an antihyperglycemic effect. Thus, the extract of the present invention can be used for oral administration for modulating the amount of free glucose in blood, thus lowering the glucemic load of a meal. The lowering effect in post-prandial blood glucose of the extract of the present invention is illustrated in Example 6.

It has been found that the buckwheat extract of the present invention is a blood glucose levels controlling agent that reduces the post-prandial glucose levels. In a preferred embodiment, the control of the blood glucose levels favors the weight controlling agent with aesthetically satisfactory results. Thus, another aspect of the present invention is the use of the buckwheat extract of the present invention as a weight controlling agent.

The non-therapeutic use of the buckwheat extract of the present invention, alone or in combination with saccharide, an iminocyclitol or probiotics, for avoiding post-prandial glycemic/insulemic imbalance is also considered that forms part of the present invention.

It has also been found that the extract can also be use in the prevention and/or coadjuvant treatment of microbiota imbalance reducing the adhesion of some potentially harmful microorganism in the microbiota and therefore increasing resistance to diseases.

Moreover, the buckwheat extract of the present invention which comprises an amount of D-fagomine between 2% and 40% by weight of dry extract mass can be used in the prevention and/or coadjuvant treatment of diabetes. This aspect could be also formulated as the use of the extract as defined above for the preparation of a medicament for the prevention and/or coadjuvant treatment of diabetes. It also relates to a method for the prevention and/or coadjuvant treatment of diabetes which comprises administering to mammals in need of such treatment an effective amount of the extract of the present invention. Thus, this effect of the extract of the present invention is shown in the results of Example 6.

It has also been reported that some sugars such as mannose (cf. Sharon et al., “Bacterial adherence to cell surface sugars”, Ciba Found Symp., 1981, vol. 80, pp. 119-41) and several iminocyclitols, such as D-fagomine, and 1-deoxynojirimycin (cf. Barira Islam et al., “Novel anti-adherence activity of mulberry leaves: Inhibition of Streptococcus mutants biofilm by 1-deoxynojirimycin isolated from Morus alba”, Journal of Antimicrobial Chemotherapy, 2008, vol. 62, pp. 751-757) inhibit the bacterial adherence to epithelial cells. There is compelling evidence showing that adherence to epithelial cells of enteric or oral bacteria is required for colonization and that colonization is required for subsequent development of symptoms of diseases. (cf. Ofek et al., “Bacterial adhesion to animal cells and tissues”, ASM Press, 2003, chapter 1). This fact makes D-fagomine useful for the prevention and/or coadjuvant treatment of a microbiota imbalance caused by enteric or oral bacteria.

The buckwheat extract of the present invention which comprises an amount of D-fagomine between 2% and 40% by weight of the extract can be used in the prevention and/or coadjuvant treatment of microbiota imbalance.

Thus, another aspect of the invention is an extract of the present invention for the prevention and/or coadjuvant treatment of a microbiota imbalance caused by enteric or oral bacteria. This aspect could be also formulated as the use of the extract as defined above for the preparation of a medicament for the prevention and/or coadjuvant treatment of a microbiota imbalance caused by enteric or oral bacteria. It also relates to a method for the prevention and/or coadjuvant treatment of a microbiota imbalance caused by an enteric or oral bacteria which comprises administering to mammals in need of such treatment an effective amount of the extract of the present invention. Thus, this effect of the extract of the present invention is shown in the results of Example 6.

D-fagomine is capable of inhibiting the adherence of enteric or oral bacteria. Enteric bacteria can be responsible for diarrhea disease or dysentery and salmonellosis among others. Examples of enteric bacteria are selected from the group consisting of Enterococcaceae and Enterobacteriaceae families. Enterococcaceae family includes, but are not limited to, Enterococcus genus such as Enterococcus faecium and Enterococcus faecalis. Enterobacteriaceae family includes, but are not limited to, Enterobacter, Escherichia, Klebsiella, Salmonella or Shigella genus. Examples of species included in this genus include, but are not limited to, Enterobacter aerogenes, Escherichia coli, Klebsiella spp., Salmonella tphimurium, Salmonella enterica or Shigella flexneri. In a preferred embodiment the enteric bacteria is selected from the group consisting of Salmonella tphimurium, and Escherichia coli.

Oral bacteria can be responsible of the two main oral disease, that is dental caries and periodontal disease. Examples of oral bacteria are selected from the group consisting Streptococcaceae family, Lactobacillaceae family, Staphylococcaceae family, Corynebacteriaceae family, and bacteria belonging to Porphyromonas genus, Aggregatibacter genus, Fusobacterium genus, and Actinomyces genus.

Streptococcaceae family includes, but is not limited to, Streptococcus genus. Examples of species of Streptococcus genus include Streptococcus mitis, Streptococcus oralis, Streptococcus sanguis, Streptococcus gordonii, Streptococcus viridans, Streptococcus mutans, Streptococcus salivarius or Streptococcus sanguis.

In a preferred embodiment the oral bacteria is selected from the group consisting of Streptococcus mutans.

In a preferred embodiment, the extract of the present invention used for the prevention and/or coadjuvant treatment of a microbiota imbalance caused by enteric or oral bacteria can be used in combination with other suitable bioactive compounds such as a saccharide, an iminocyclitol or probiotics. Thus, the functional food, dietary supplement, pharmaceutically or veterinary, as defined above in combination with other suitable bioactive compounds such as a saccharide, a iminocyclitol or probiotics are also part of the invention.

Suitable saccharides useful for the present invention are selected from the group consisting of alpha-methyl-D-mannoside, globotetraose, mannose, gal α(1 4) gal o linked methyl, sialyl 3′ lactose, sialyl-gal (β 1 4)GlcNAc, oxidized carbohydrate alpha (1,6)-glucans.

Suitable iminocyclitols that inhibit the bacterial adherence to epithelial cells useful for the present invention are selected from the group consisting of, deoxynojirimycin, miglitol, and miglustat.

Suitable probiotics useful for the present invention are selected from the group consisting of microorganism of the Lactobacillaceae family, including genus Lactobacillus, and Bifidobacteriaceae family, including genus Bifidobacterium. Examples of species of suitable probiotics includes, but are not limited to, Lactobacillus rhamnosus, Lactobacillus salivarius, Lactobacillus plantarum, Lactobacillus acidophilus, Lactobacillus casei, Bifidobacterium bifidum, Bifidobacterium longum, Bifidobacterium infantis, or Lactococus lactis.

Throughout the description and claims the word “comprise” and variations of the word, are not intended to exclude other technical features, additives, components, or steps. Additional objects, advantages and features of the invention will become apparent to those skilled in the art upon examination of the description or may be learned by practice of the invention. The following examples and drawings are provided by way of illustration, and they are not intended to be limiting of the present invention. Reference signs related to drawings and placed in parentheses in a claim, are solely for attempting to increase the intelligibility of the claim, and shall not be construed as limiting the scope of the claim. Furthermore, the present invention covers all possible combinations of particular and preferred embodiments described herein.

### EXAMPLES

### Example 1

### Purification of D-Fagomine by Mashing Buckwheat Meal with Exogenous Enzymes and Fermentation of the Wort Followed by Ion-Exchange Resins

### Step 1. Milling

10 kg of buckwheat seeds were milled in a continuous milling/sieving device model MXAS from Iberital (Sant Feliu de Llobregat, Spain). The resulting buckwheat meal had an average particle size of 0.75 mm.

### Step 2. Mashing

The buckwheat meal (3 kg) was mixed with 9 L of water at 54° C. in the presence of a cocktail of three different enzymes: BAN 240 (Novozymes, Bagsvaerd, Denmark) (α-amilase, 24 g) which brakes 1→4 internal bonds, Neutrase 0.8 (Novozymes) (proteinase, 3 mL) and Viscoflow MG (Novozymes) (mixture of beta-glucanase, xylanase and a wide range of carbohydrase enzymes, 0.45 g). The temperature was increased to 70° C. in about 30 min and the mixture was kept at 70° C. for 30 min. Then, the temperature was decreased to 55° C. Next, two more enzymes were added to break the dextrins: Promoenzyme BrewQ (Novozymes) (pululanase, 4 mL) and AMG 300 BrewQ (Novozymes) (amiloglucosidase, 10 mL).

### Step 3. Boiling and Centrifugation

The wort obtained in the previous step was kept at 55° C. for 60 min. Then, it was passed through a 0.5 mm mesh, the volume adjusted to 10 L and boiled for 15 min. 3 L of the boiled wort were centrifuged at 8000 g for 5 min at 20° C. in a 4K15 centrifuge from Sigma (Buckinghamshire, England). The sediment was discarded by decantation and 2.1 L of boiled wort were obtained.

### Step 4. Fermentation

2.1 L of boiled wort were inoculated with 0.8 g of dehydrated yeasts Safale US-05 (Fermentis, Marcq-en-Baroeul, France) (11.5 g pack of dehydrated yeasts for 30 L boiled wort). The fermentation was carried out in a thermostated chamber at 14° C. for 10 days. The resulting beer-like fermented mixture (FM) was centrifuged at 3000 g for 5 min at 20° C. in a 4K15 centrifuge from Sigma (Buckinghamshire, England) and the residue discarded.

### Step 5. Purification by a Cation-Exchange Resin (IMAC)

IMAC HP336 resin (Rohm and Hass, Chauny, France), 0.1 L were packed into a 500×27 mm i.d. glass column and washed with 2 volumes of 1M ammonia in 30% ethanol and then with 40 volumes of 30% ethanol. Then the beer-like FM (1 L) was loaded into the column and the non-retained materials (e.g. non-fermentable saccharides, polyphenols) eluted with 30% ethanol (3 bed volumes) followed by 0.04 M ammonia in 30% ethanol (3 bed volumes). Then, D-fagomine was desorbed with 0.35 M ammonia in 30% ethanol (5 bed volumes). The resin was then washed with 3 bed volumes of 1 M ammonia in 30% ethanol and 40 bed volumes of 30% ethanol. The ammonia was eliminated by evaporation from the D-fagomine containing fraction and the mixture freeze-dried.

This process yields 278 mg extract/kg buckwheat.

### Step 6. Purification by an Anion-Exchange Resin (IRA)

Amberlite IRA 458 resin (Rohm and Hass, Chauny, France), 0.1 L were packed into a 500×27 mm i.d. glass column and washed with 2 volumes of 1 M NaOH and, then, with water until neutral pH. The D-fagomine containing fraction from the previous step was loaded into the column and the resin was washed with one bed volume of water. The D-fagomine containing solutions, namely the excluded load eluate and the washing eluate were pooled and freeze-dried. The IRA resin was cleaned with 1M NaOH (one bed volume) and water until neutral pH.

This process yields 21 mg extract/kg buckwheat.

The total sugars, reducing sugars, proteins and D-fagomine in wort, boiled wort, and fermented mixture were determined (Table 1). Total sugar content and reducing sugars content were calculated by the Luff-Schoorl method (International Fruit Juice Union, 1968. Determination of sugar (Luft-Schoorl method). Method n. 4). Protein was calculated by Dumas method (AOAC (Association of Official Analytical Chemists), 2000. Official method 968.06. Protein (crude) in animal feed. Dumas method. Official Methods of Analysis, 1. 17th ed. Gaithersburg, Md., USA). D-fagomine content was calculated by HPLC-MS method. The results were expressed as *%: *%: by weight of dry extract mass.

TABLE 1
Total sugars, reducing sugars, proteins and D-fagomine
in wort, boiled wort, and fermented mixture

| | Wort (*%) | Boiled wort (*%) | FM (*%) |
|---|---|---|---|
| Total sugars | 96.8 | 108 | 66.3 |
| Proteins | 13.7 | 5.80 | 7.25 |
| Reducing sugars | 47.0 | 70.2 | 29.9 |
| D-fagomine | 0.005 | 0.005 | 0.009 |

*%: by weight of dry extract mass

Wort boiling does not affect D-fagomine concentration and fermentation increases D-fagomine concentration in the dried extract. Mashing releases reducing sugars that are consumed during fermentation. Wort boiling lowers protein content.

The purification of D-fagomine from non-fermentable galactose-derived compounds such fagopyritols was monitored by MALDI-TOF which recorded the elimination of galactose polymers. Freeze-dried samples (1 mg) were dissolved in water (1 mL). A solution of DHB (2,5-Dihydroxybenzoic acid) Sigma Aldrich (St Louis, Mo., USA) (MALDI matrix, 1 μL of a 10 mg/mL solution) was spotted onto a MALDI plate and let dry. Then the sample solution (1 μL) was spotted over the dried DHB, (2,5-dihydroxybenzoic acid) and let dry.

Analysis conditions were: System, Autoflex III Smartbeam MALDI/TOF from Bruker (Billerica, Mass., USA); mass range, 400-1900; 2100-4500; mode positive; ion source 1, 19 kV; ion source 2, 16.36 kV; lens, 8.60 kV; reflector 1, 21 kV; reflector 2, 16.36 kV; electronic gain, 100 mV; laser attenuator, 50%; detector gain (reflector detector voltage), 1611 V; shots, 500; frequency, 200.

Compounds such as fagopyritols, putatively containing units of galactose show the following theoretical masses.

| Units | Compound + Na | Compound + K |
|---|---|---|
| 2 | 365.292 | 381.401 |
| 3 | 527.436 | 543.544 |
| 4 | 689.580 | 705.688 |
| 5 | 851.723 | 867.832 |
| 6 | 1013.867 | 1029.975 |
| 7 | 1176.010 | 1192.119 |
| 8 | 1338.154 | 1354.262 |
| 9 | 1500.298 | 1516.406 |
| 10 | 1662.441 | 1678.550 |
| 11 | 1824.585 | 1840.693 |
| 12 | 1986.728 | 2002.837 |
| 13 | 2148.872 | 2164.980 |
| 14 | 2311.016 | 2327.124 |
| 15 | 2473.159 | 2489.268 |
| 16 | 2635.303 | 2651.411 |
| 17 | 2797.446 | 2813.555 |
| 18 | 2959.590 | 2975.698 |
| 19 | 3121.734 | 3137.842 |
| 20 | 3283.877 | 3299.986 |
| 21 | 3446.021 | 3462.129 |
| 22 | 3608.164 | 3624.273 |
| 23 | 3770.308 | 3786.416 |
| 24 | 3932.452 | 3948.560 |
| 25 | 4094.595 | 4110.704 |
| 26 | 4256.739 | 4272.847 |

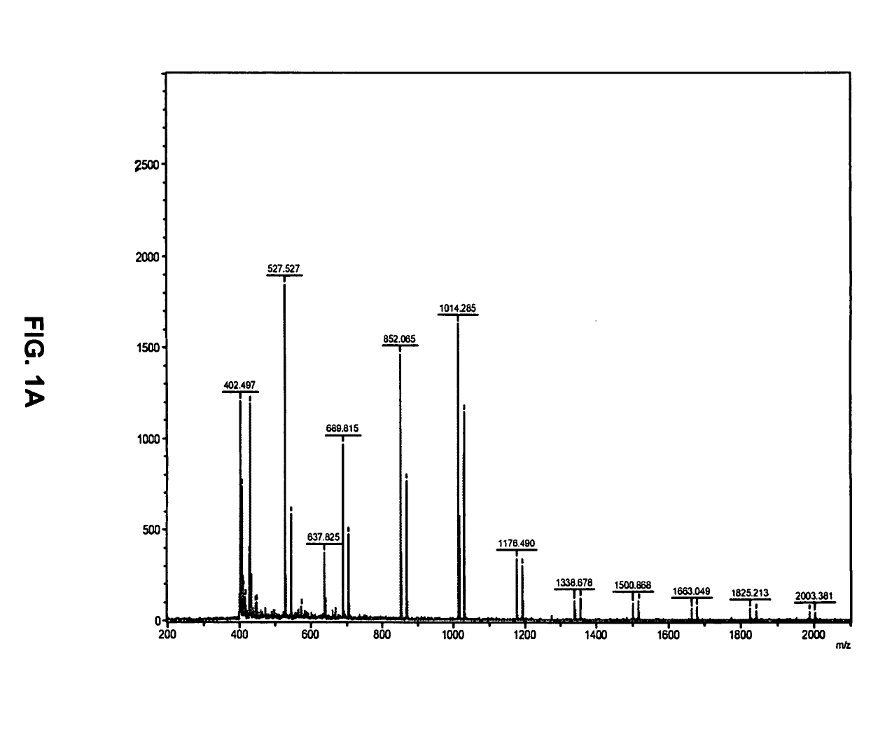

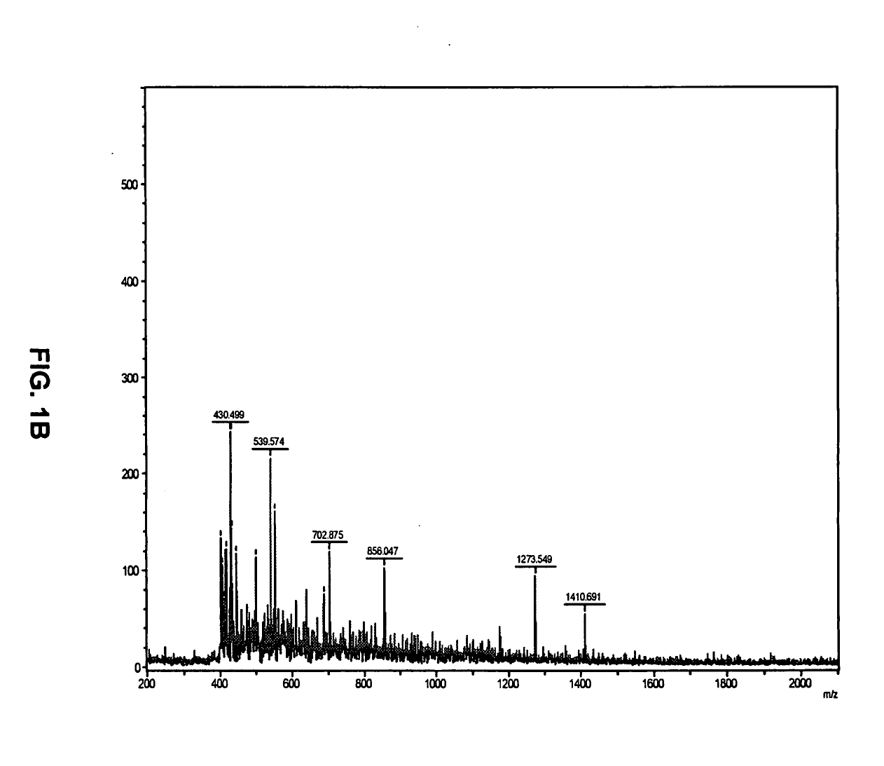

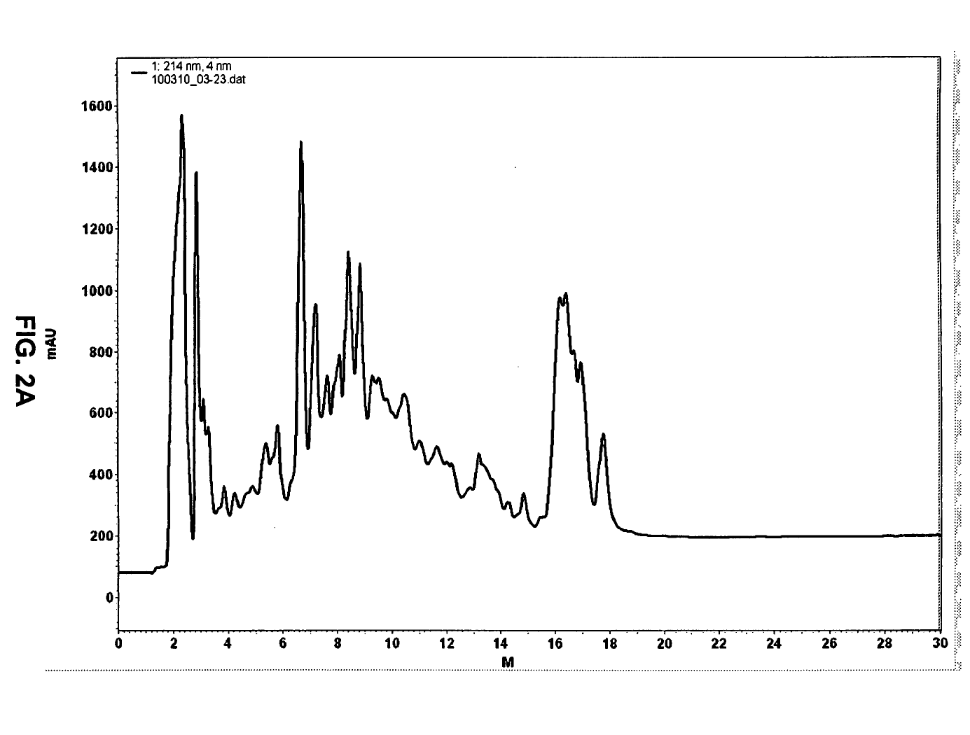

FIG. 1 shows the MALDI-TOF (Matrix-Assisted Laser Desorption/Ionization-Time-Of-Flight) analysis results of the non-retained materials in cation exchange resin (A) and the retained materials (B) from Example 1. IMAC resin retained D-fagomine while non-fermentable polysaccharides including galactose-derived compounds such as fagopyritols were excluded.

### Example 2

### Purification of D-Fagomine by Mashing Buckwheat with Barley Malt Meal and Fermentation of the Wort Followed by Ion-Exchange Resins

### Step 1. Milling

1.5 kg of buckwheat (seeds) and 1.2 Kg of barley malt were milled in a continuous milling/sieving device model MXAS from Iberital (Sant Feliu de Llobergat, Spain). The resulting buckwheat and barley malt meal had an average particle size of 0.75 mm.

### Step 2. Mashing

The buckwheat and barley malt meal (2.7 kg) was mixed with 8.1 l of water and kept at 40° C. for 30 min. The temperature was increased to 60° C. in about 30 min and the mixture was kept at 60° C. for 30 min. Then, the temperature was increased to 70° C. in about 5 min and the mixture kept at this temperature for 30 min.

Next, temperature was increased to 70° C. in about 5 min and kept at this temperature for 30 min.

### Step 3. Boiling

The wort obtained in the previous step was passed through a 0.5 mm mesh and boiled for 60 min, the volume adjusted to 10 L and boiled for 15 min. 3 L of the boiled wort were centrifuged at 8000 g for 5 min at 20° C. in a 4K15 centrifuge from Sigma (Buckinghamshire, England). The sediment was discarded by decantation.

### Step 4. Fermentation

1 L aliquots were taken, inoculated with 0.4 g of dehydrated yeast Safale US-05 (Fermentis, Marcq-en-Baroeul, France) and fermented following the description in Example 1.

TABLE 2

| | Wort (%)* | Boiled wort (%)* | FM (%)* |
|---|---|---|---|
| Total sugars | 124 | 116 | 7.0 |
| Reducing sugars | 110 | 108 | 6.9 |
| Proteins | 0.05 | 0.04 | 3.2 |
| D-fagomine | 0.002 | 0.002 | 0.005 |

*%: by weight of dry extract mass.

Table 1 and Table 2 show that mashing with endogenous or exogenous enzymes produce starch breakdown and the reducing sugar release necessary to carry out the next fermentation step. In both cases with endogenous and/or exogenous enzymes, dry mass is reduced up to 40% after sugar consumption by fermentation and, consequently, is D-fagomine enriched.

Boiling is not essential for any Example 1 or 2.

### Example 3

### Purification of D-Fagomine by Fermentation of Modified Buckwheat Wort Followed by Ion-Exchange Resins

The process described in Example 1 was modified and improved by the inclusion of an adsorption step after the preparation of the boiled wort and immediately before fermentation.

### Steps 1, 2 and 3.

These steps were identical to the ones described for Example 1.

### Step 4. Purification by Adsorption Resin

The boiled wort from Example 1, step 3 (2.1 L) was processed by an absorption resin. FPX 66 resin (Rohm and Haas, Chauny, France), 0.4 L were packed into a 600×80 mm i.d. glass column and washed with 5 volumes of ethanol and then 10 volumes of water. Then, the boiled wort (0.7 L) was loaded into the column and the non-retained material which contains the fermentable sugars, non-fermentable carbohydrates (e.g. fagopyritols) and D-fagomine were eluted with 0.5 bed volumes of water to obtain a final volume of 0.9 L of D-fagomine solution. The column was freed from the retained material (e.g. proteins and polyphenols) by washing with 4 bed volumes of ethanol and re-conditioned with 7.5 bed volumes of water. The process was repeated (2×0.7 L boiled wort). The solutions containing D-fagomine were pooled.

### Step 5. Fermentation

The previously processed boiled wort (2.7 L) was inoculated with 1 g of dehydrated yeasts Safale US-05 (Fermentis, Marcq-en-Baroeul, France) (11.5 g pack of dehydrated yeasts for 30 L boiled wort). The fermentation was carried out in a thermostated chamber at 14° C. for 10 days. The resulting FM was centrifuged at 3000 g for 5 min at 20° C. in a 4K15 centrifuge from Sigma (Buckinghamshire, England) and the residue was discarded.

To maximize the fermentation yield and consequently the degree of purification of D-fagomine, the product from step 4 may be concentrated to one half of its volume.

### Step 6. Purification by a Cation-Exchange Resin (IMAC)

IMAC HP336 resin (Rohm and Hass, Chauny, France), 0.1 L is packed into a 500×27 mm i.d. glass column and washed with 2 volumes of 1 M ammonia in 30% ethanol and then with 40 volumes of 30% ethanol. Then the beer-like FM from step 5 (1 L) was loaded into the column and the non-retained materials (e.g. non-fermentable saccharides, including fagopyritols) was eluted with 30% ethanol (3 bed volumes) followed by 0.04 M ammonia in 30% ethanol (3 bed volumes). Then, D-fagomine was desorbed with 0.35 M ammonia in 30% ethanol (5 bed volumes). The resin was then washed with 3 bed volumes of 1 M ammonia in 30% ethanol and 40 bed volumes of 30° A ethanol. The ammonia was eliminated by evaporation from the D-fagomine containing fraction and the mixture freeze-dried.

This process yields 46 mg extract/kg buckwheat.

### Step 7. Purification by an Anion-Exchange Resin

This step was identical to the one described for Example 1.

This process yields 12 mg extract/kg buckwheat.

Total proteins and D-fagomine were determined in wort, boiled wort, and fermented mixture (Table 2). Protein was calculated by the Dumas method (AOAC (Association of Official Analytical Chemists), 2000. Official method 968.06. Protein (crude) in animal feed. Dumas method. Official Methods of Analysis, 1. 17th ed. Gaithersburg, Md., USA) D-fagomine content was determined by the HPLC-MS method (Example 4). The results were expressed as % by weight of extract mass.

TABLE 2
Proteins and D-fagomine in wort, boiled
wort, and fermented mixture

| | Wort (%)* | Boiled wort (%)* | FM (%)* |
|---|---|---|---|
| Proteins | 13.7 | 5.80 | 2.48 |
| D-fagomine | 0.005 | 0.005 | 0.008 |

*%: by weight of dry extract mass.

The purification by adsorption resin step reduces protein concentration in the extract.

Samples were analyzed by HPLC-UV to monitor the elimination of proteins and polyphenols at a wavelength of 214 nm: boiled wort (FIG. 1, A), a sample after the purification by adsorption resin (FIG. 1, B) and a sample after the fermentation step (FIG. 1, C). The system used was Hitachi Elite LaChrom from VWR (Philadelphia, Pa., USA). The detector used was DAD from VWR (Philadelphia, Pa., USA). The column used was Kromasil 100 C₁₈ 5 μm (50×4.0 mm i.d.) from Teknokroma (Barcelona, Spain). The solvent in the mobile phase used were A:0.1% aqueous trifluoroacetic acid, B:0.1% trifluoroacetic acid in CH₃CN, gradient, 10 to 80% in 30 min. The flow was 1 mL/min and the column temperature was 30° C.

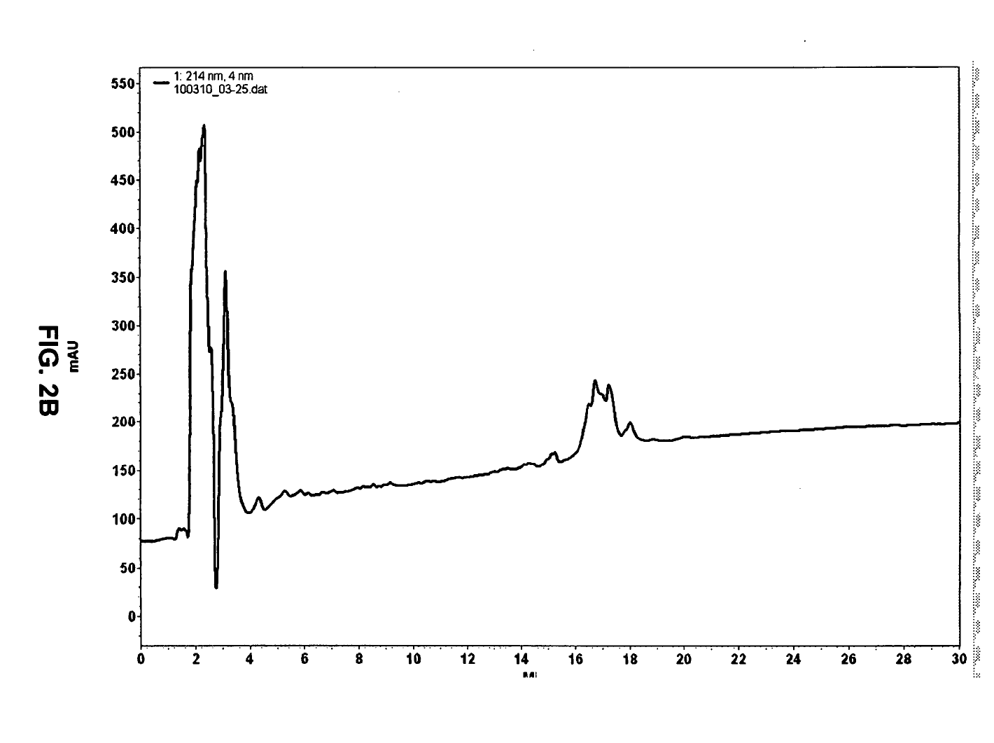

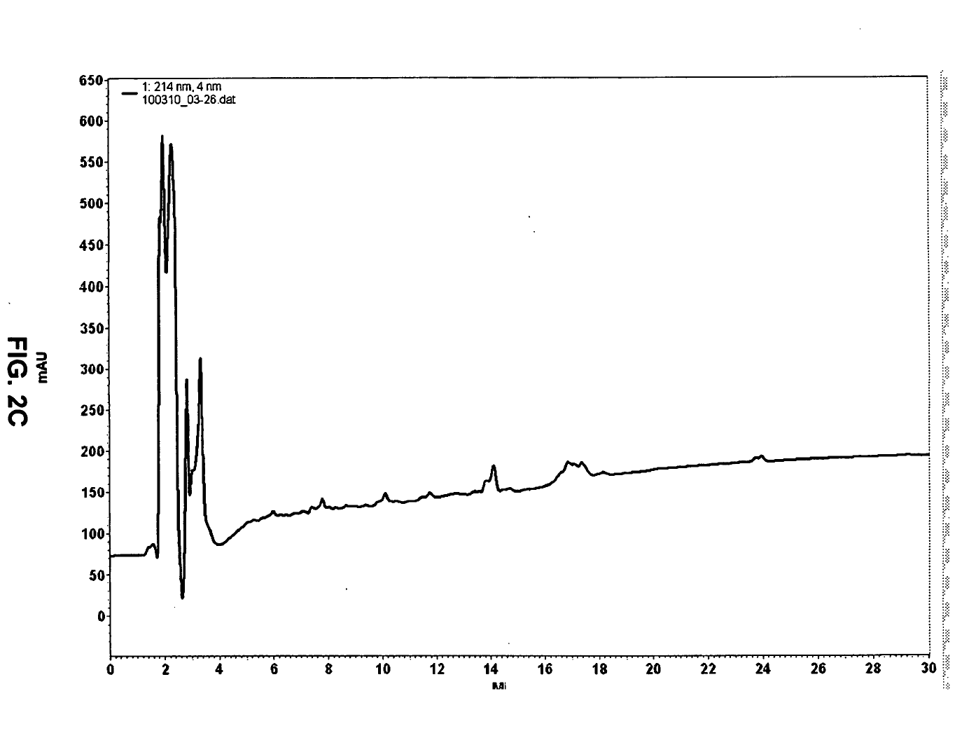

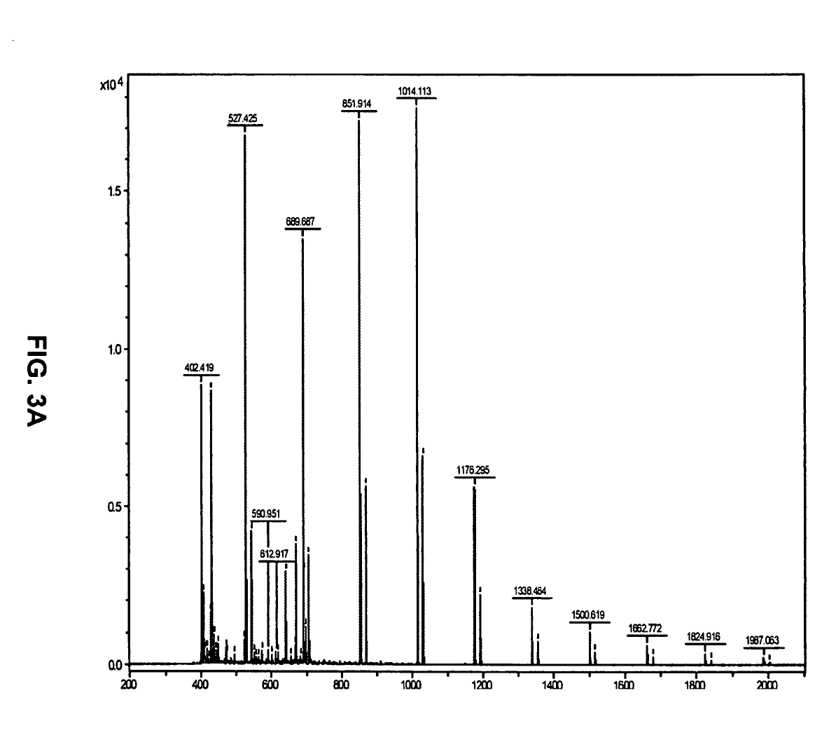

FIG. 2 shows how the FPX resin retains hydrophobic compounds including proteins/peptides and polyphenols. Most of the proteins, peptides and polyphenols were retained in the FPX resin, some material not retained by the resin may be composed of bulky proteins excluded from the resin which are subsequently eliminated in the fermentation step.

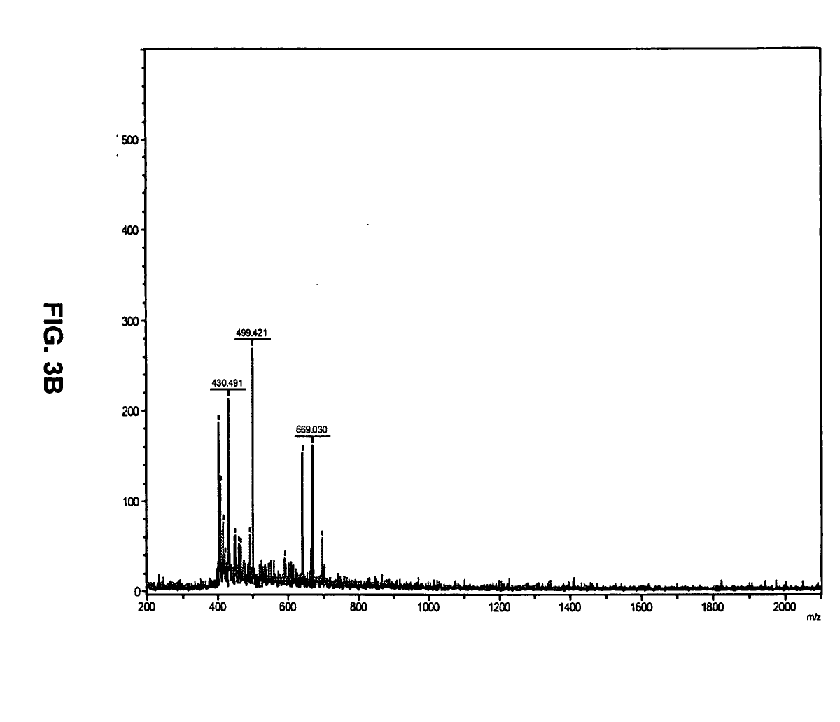

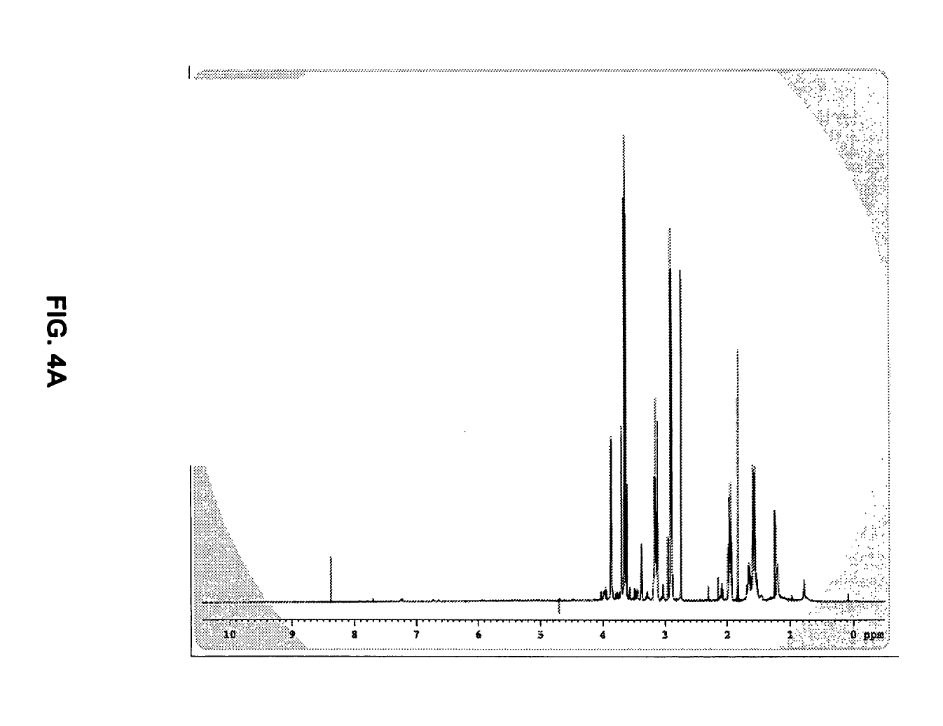

The purification of D-fagomine from non-fermentable galactose-derived compounds such fagopyritols was monitored by MALDI-TOF as described in Example 1. FIG. 3 shows that IMAC resin retains D-fagomine while non-fermentable polysaccharides including fagopyritols are excluded.

### Example 4

### Purification of D-Fagomine by Fermentation of Modified Buckwheat Wort Followed by Ion-Exchange Resins Under Improved Elution Conditions

The process described in Example 3 was improved by modifying the elution conditions in steps 4 and 6.

### Steps 1, 2, 3.

These steps were identical to the ones described for Example 3.

### Step 4. Purification by Adsorption Resin

FPX 66 resin (Rohm and Haas, Chaney, France), 0.25 L were packed into a 500×48 mm i.d. glass column and washed with 5 volumes of ethanol and then 10 volumes of water. Then, the boiled wort (225 mL) was loaded into the column and the non-retained material which contains the fermentable sugars, non-fermentable carbohydrates (e.g. fagopyritols) and D-fagomine were eluted with a bed volume of water. The column was freed from the retained material (e.g. proteins and polyphenols) by washing with 4 bed volumes of ethanol and re-conditioned with 7.5 bed volumes of water. The process was repeated with a second aliquot (225 mL) of boiled wort. The solutions containing D-fagomine were pooled (0.95 L).

### Step 5. Fermentation

The previously processed boiled wort (0.95 L) was inoculated with 0.36 g of dehydrated yeasts Safale US-05 (Fermentis, Marcq-en-Baroeul, France) (11.5 g pack of dehydrated yeasts for 30 L boiled wort). The fermentation was carried out in a thermostated chamber at 14° C. for 10 days. The resulting FM was centrifuged at 3000 g for 5 min at 20° C. in a 4K15 centrifuge from Sigma (Buckinghamshire, England) and the residue discarded.

To maximize the fermentation yield and consequently the degree of purification of D-fagomine, the product from step 4 may be concentrated to compensate for the dilution in the resin step 4.

### Step 6. Purification by a Cation-Exchange Resin (IMAC)

IMAC HP336 resin (Rohm and Hass, Chauny, France), 68 mL were packed into a 500×27 mm i.d. glass column and washed with 2 volumes of 1 M ammonia in 30% ethanol and then with 40 volumes of 30% ethanol. Then the beer-like FM from step 5 (0.68 L) was loaded into the column and the non-retained materials (e.g. non-fermentable saccharides, including fagopyritols) eluted with 30% ethanol (4 bed volumes) followed by 0.04 M ammonia in 30% ethanol (3 bed volumes). Then, D-fagomine was desorbed with 1 M ammonia in 30% ethanol (5 bed volumes). The resin was then washed with 2 bed volumes of 1 M ammonia in 30% ethanol and 40 bed volumes of 30% ethanol. The ammonia was eliminated by evaporation from the D-fagomine containing fraction and the mixture freeze-dried.

This process yields 107 mg extract/kg buckwheat.

### Step 7. Purification by an Anion-Exchange Resin

This step was identical to the one described for Example 1.

This process yields 21 mg extract/kg buckwheat.

### Example 5

### Determination Of the Amount of D-Fagomine and 3,4-di-epi Fagomine in Example 1, 3, and 4

The amount of D-fagomine and its isomer were determined by HPLC-MS and expressed by weight of dry extract mass.

The HPLC-MS analysis was carried out using an optimized protocol.

### HPLC-MS Protocol

### Extraction:

500 μL of sample were spiked with 70 μL of a 100 mg/L solution of DMDP (2,5 dideoxy-2,5-imino-D-mannitol) from IRL (Lower Cut, New Zealand) (internal standard) and mixed with methanol (7 mL) at −20° C. and water (2 mL). The sample was kept at −20° C. for 30 min and then, it was filtered through a 0.45 μm 25 mm nylon filter (Afora, Barcelona, Spain).

### Purification:

Analytes purification was carried out by solid phase extraction with SCX (Applied separations, Allentown, Pa., USA) cartridges washed with 1 mL of HPLC grade methanol, equilibrated with 1 mL of HPLC grade water. The samples were loaded and the cartridge rinsed with water (1 mL) and eluted with 450 μL of NH₃ 2 M in HPLC grade water. The solvent was evaporated to dryness in a 60° C. bath under nitrogen flow. The residue was suspended in 400 μL of HPLC grade water and filtered through a Millex PHV 0.45 μm 13 mm (Millipore, Barcelona, Spain) filter.

### Analysis:

The following table describes the HPLC conditions: chromatographic column, TSK-Gel CM-2SW (4.6 mm×25 cm, 5 μm) from Tosoh Bioscience (Tokio, Japan); solvents A: NH₃ 50 mM pH=8.3 (adjusted with acetic acid), B:CH₃OH; mobile phase, isocratic, 20% B, flow 0.8 mL/min; injection volume, 20 μL; columns temperature, 25° C.; analysis time, 30 min.

The MS equipment was a TSQ 7000 from Thermoscientific (est Palm Beach, Fla., USA). The MS conditions were: analyzer, simple quadrupole; ionization, electrospray; capillary voltage, 4.5 kV; capillary temperature, 250° C.; gas N₂, 60 psi; auxiliary gas N₂, 30 psi; multiplier, 1350V; analysis mode, SIM.

Under these conditions, D-fagomine and a fagomine isomer yield [M+1]⁺ signals at m/z=148.2 (t_{R}=16 min and 25 min, respectively). DMDP, the internal standard, yields a [M+1]⁺ signal at m/z=164.2 (t_{R}=8 min).

### Determination:

To quantify the amount of D-fagomine and fagomine isomer in the samples, a calibration curve was generated with D-fagomine standards in the range 1-10 mg/L. These standards contained the necessary volumes of 20 mg/L of D-fagomine standard, 70 μL of a 100 mg/L solution of DMDP and HPLC grade water up to 400 μL of total volume.

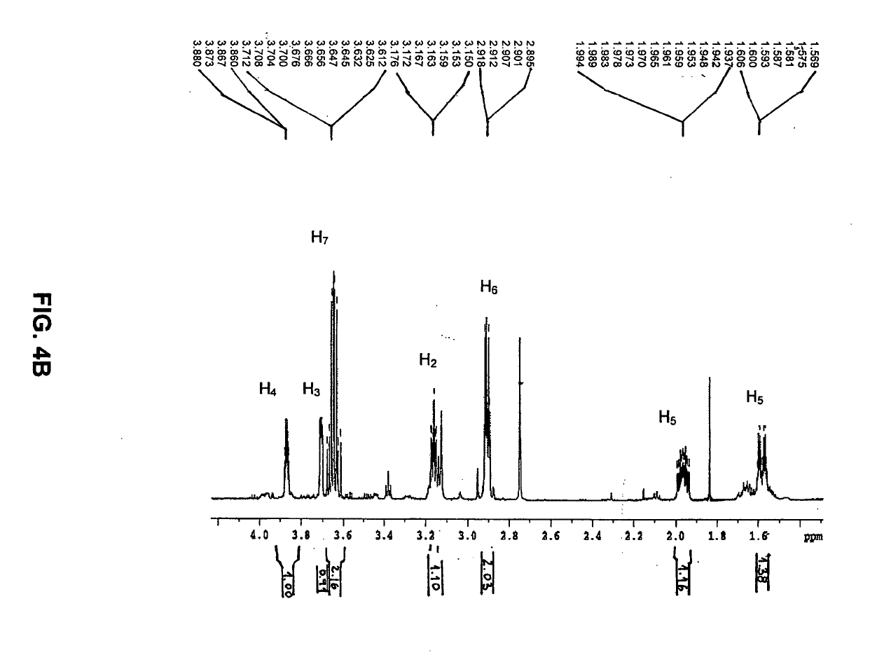

The structure of the compound at m/z 148.2, t_{R}=25 min was elucidated after purification. Buckwheat was extracted with an aqueous solution of methanol and the compounds were fractionated with a CM Sepharose Fast Flow (GE Healthcare, Uppsala, Sweeden) column in a FPLC system (Pharmacia Biotech, Uppsala, Sweeden). Fractions were freeze-dried and analyzed by HPLC-MS. Fractions containing the compound at t_{R}=25 min were pooled and analyzed in an Avanced 2 Plus 600 NMR system (Bruker). The NMR assignments are compatible with 3,4-di-epifagomine or its enantiomer (2-epi-fagomine). FIG. 4 shows the results.

The amount of D-fagomine and 3,4-di-epifagomine was determined by HPLC-MS and expressed by weight of dry extract mass in the different steps of the process of the invention.

TABLE 3
Amount of D-fagomine determined by HPLC-MS
and expressed by weight of dry extract mass

| | Fermented | IMAC purified |
|---|---|---|
| | product (%*) | product (%*) |
| Example 1, no adsorption step before | 0.009 | 0.96 |
| fermentation | | |
| Example 3, adsorption step before | 0.008 | 3.58 |
| fermentation | | |

*%: by weight of dry extract mass.

IMAC resin concentrates D-fagomine. Previous FPX purification allows a more efficient IMAC purification because it eliminates material (e.g. peptides/proteins, polyphenols) that occupies reactive sites on the resin surface by ion-exchange and/or hydrophobic interactions.

TABLE 4
Amount of D-fagomine is determined by HPLC-MS
and expressed by weight of dry extract mass

| | | IMAC | IRA |
|---|---|---|---|
| | Fermented | purified | purified |
| | product | product | product |
| | (%*) | (%*) | (%*) |
| Example 3, adsorption step before | 0.008 | 3.58 | 8.96 |
| fermentation | | | |
| Example 4, adsorption step before | 0.010 | 3.64 | 18.3 |
| fermentation improved IMAC | | | |

*%: by weight of dry extract mass.

IRA resin concentrates D-fagomine in the extract. In Example 4 IMAC purification has been optimized, with subsequent improvement of the final purity. The yield was also improved (Table 5).

TABLE 5
mg extract/kg initial buckwheat

| | IMAC purified | IRA purified |
|---|---|---|
| mg extract/kg initial buckwheat | product | product |
| Example 3, adsorption step before | 46 | 12 |
| fermentation | | |
| Example 4, adsorption step before | 107 | 21 |
| fermentation improved IMAC | | |

At all times in the purification process, the stereoisomer of D-fagomine has been co-purified with D-fagomine. The NMR assignments are compatible with 3,4 Di-epifagomine or its enantiomer 2-epi-fagomine. The results may be given as amount of both isomers where the isomer is expressed as D-fagomine equivalents (Table 6).

TABLE 6
Amount of D-fagomine and the putative 3,4-di-epifagomine, determined
by HPLC-MS and expressed by weight of dry extract mass.

| | | D-fagomine + |
|---|---|---|
| | D-fagomine (%*) | diastereomer (%*) |
| Example 1 | 12% | 18% |
| Example 3 | 9% | 13% |
| Example 4 | 18% | 31% |

*%: by weight of dry extract mass.

The following table 7 shows the total fagomine equivalents in the extract and previous steps in the preparation of the extract.

TABLE 7
Total fagomine equivalents

| | Example 3 (%*) | Example 4 (%*) |
|---|---|---|
| Wort | 0.010 | 0.010 |
| Boiled wort | 0.010 | 0.010 |
| Fermented mixture | 0.016 | 0.018 |
| Mixture after IMAC | 5.44 | 6.00 |
| Final extract after IRA | 13.4 | 31.2 |

*%: by weight of dry extract mass.

### Example 6

### Post-Prandial Blood Glucose Test

The effect on postprandial blood glucose of both D-fagomine enriched extract from buckwheat and D-fagomine obtained chemoenzimatically following the procedure described in WO2008025826A1 was evaluated.

Adult male Sprague-Dawley rats of 200-220 g body weight (Janvier, Le Genest-St-Isle, France) were housed in cages (n=2/cage) under controlled conditions of stable humidity (40-77%), and temperature (23° C.) with a 12-hour light/dark cycle. The rats were fed a standard diet (Panlab A04, Panlab, Barcelona, Spain) and given water ad libitum. To minimize circadian rhythm effects, rat manipulations were carried out in the morning. Handling and sacrificing of the animals were in full accordance with the European Union guidelines for the care and management of laboratory animals and the pertinent permission was obtained from the CSIC Subcommittee of Bioethical Issues. The authors further attest that all efforts were made to minimize the number of animals used and their suffering.

The glucose test was performed after a 12 h food deprivation period. A solution of sucrose (2 g/kg body weight) together with the appropriate amount of the compound being tested was administered to the rats. Negative and positive control experiments were performed by administration of water or sucrose solution, respectively. A dose of 1.0 mg kg⁻¹ body weight of D-fagomine coming from the buckwheat extract (8.4 mg extract mL⁻¹) and D-fagomine standard and the controls were administered as water solutions (5 mL kg⁻¹ body weight) using a gastric probe. Blood samples were collected from the saphenous vein (14) at 0, 15, 30, 45, 60, 90 and 120 min after administration. Blood glucose concentration was measured by the enzyme electrode method using Elite blood glucose test strips and a blood glucose meter Ascensia ELITE XL both from Bayer Consumer Care AG (Basel, Switzerland). The areas under the curve (AUC) up to 120 min were calculated according to the trapezoidal rule using Graph Pad Prism 4.

TABLE 8
Post-prandial blood glucose after administration
of D-fagomine extract from buckwheat and pure
D-fagomine obtained chemoenzymatically

| | Blood glucose (mg/mL) after administration of | | | |
|---|---|---|---|---|
| | | | Sucrose + | Sucrose + |
| Time | | | D-fagomine, | D-fagomine |
| (min.) | Water | Sucrose | Example 1 | WO2008025826A1 |
| 0 | 49 | 54 | 41 | 51 |
| 15 | 67 | 112 | 75 | 79 |
| 30 | 66 | 106 | 86 | 91 |
| 45 | 62 | 98 | 86 | 93 |
| 60 | 62 | 80 | 83 | 83 |
| 90 | 65 | 84 | 76 | 76 |
| 120 | 58 | 70 | 59 | 62 |
| 180 | 45 | 59 | 60 | 61 |
| 240 | 56 | 60 | 56 | 59 |

The increase in plasma glucose after sucrose administration was lowered by D-fagomine. Extracted and chemoenzymatic D-fagomine were equally effective.

### Example 7

### Adhesion of Escherichia coli to Mucus in the Presence of D-Fagomine

Adhesion to Mucus. Protocol

The strains of Escherichia coli used were obtained from the Bacterial Strain Collection of the Faculty of Veterinary Science at the Universitat Autònoma de Barcelona. Overnight, cultures of the bacterial strain were inoculated into flasks containing Luria medium (Liofilchem, Roseto degli Abruzzi, Italy) (3 mL) to facilitate the production of fimbriae. The strains were incubated at 37° C. for 24 h. Then, colonies were grown on Luria medium Agar plates and after 24 h of incubation, several dilutions were prepared in PBS (phosphate buffered saline, Sigma Aldrich, St Louis, Mo., USA) for each strain, down to a concentration of 1×10⁷ CFU mL⁻¹.

The mucosa was obtained from intestinal segments of pigs, just after they were killed at a local slaughterhouse, and kept frozen until use. The mucosa preparation (1 mL) was defrosted, centrifuged and mixed with PBS (99 mL). Prior to each test, the concentration of mucine in the suspension was calculated by the Bradford method. Multiwell plates (Nunc®, Roskilde, Denmark) were treated with the mucosa suspension (2.5 mL) overnight at 4° C. Then the suspensions were carefully sucked off and the wells gently loaded with bacterial suspensions (1×10⁷ CFU mL⁻¹) followed immediately by the addition of the appropriate amount of D-fagomine coming from the enriched buckwheat extract to reach the desired final concentrations (10 mg L⁻¹, 0.07 mmol L⁻¹) in a total volume of 1.5 mL. The mixtures were incubated at 37° C. for 90 min. D-fagomine standard 0.07 mmol L⁻¹ was used as a control and the experiment was performed in triplicate. Finally, the colony forming units from both the supernatant and mucosa were counted under an optical microscope after cultivating both fractions overnight.

TABLE 3
Mucus recounted with D-fagomine extract from buckwheat
and pure D-fagomine obtained chemoenzymatically 10 mg/L

| | Mucus CFU/ml |
|---|---|
| Control | |
| Escherichia coli | 2.09 10¹⁰ |
| D-fagomine extract Example 1, 10 mg/L | |
| Escherichia coli | 6.83 10⁹ |
| D-fagomine WO2008025826A1 10 mg/L | |
| Escherichia coli | 6.70 10⁹ |

The microorganisms were detected in significant amounts only in the supernatant, not in the mucus. The microorganisms were agglutinated by D-fagomine obtained from buckwheat or a chemoenzymatic process. Both were equally effective and bacterial adhesion to the mucus was not detected.

### Example 8

### Weight Ratios of 3,4-di-Epifagomine/D-Fagomine

Initial weight ratio of 3,4-di-epifagomine/D-fagomine in buckwheat seeds can vary in the range comprised between 1:1-1:10. Several samples of buckwheat seeds have been analyzed to determine the weight ratio of 3,4-di-epifagomine/D-fagomine.

Samples A and E are buckwheat seeds obtained from local producers. Samples B, C and D are buckwheat seeds of the same brand marketed in Spain.

Seeds were milled using a Moulinex (Ecully Cedex, France) A 505 2HF mill. Then the milled samples (100 mg) were spiked with 70 μL of a methanolic solution containing 100 mg L⁻¹ DMDP and left semi-covered overnight until the complete evaporation of the solvent. D-Fagomine and its isomer were extracted with 70% aqueous methanol (12 mL) using an orbital shaker Intelli-mixer RM-2 (Elmi, Riga, Latvia) for 15 min. The extracts were centrifuged, filtered using a 0.45 μm nylon filter and the filtrates loaded onto SCX cartridges. Purification and analysis were performed as described in Example 5.

The content of 3,4-di-epifagomine and D-fagomine of the analyzed samples are summarized in Table 4:

TABLE 4

| | D-fagomine | 3,4-di-epifagomine | Weight ratio |
|---|---|---|---|
| | content | content | 3,4-di-epifagomine/ |
| | (mg/kg) | (mg/kg) | D-fagomine |
| Sample A | 43 | 44 | 1:1 |
| Sample B | 36 | 31 | 1:1.2 |
| Sample C | 22 | 7.3 | 1:3 |
| Sample D | 29 | 23 | 1:1.3 |
| Sample E | 18 | 2.8 | 1:6 |

### REFERENCES CITED IN THE APPLICATION

1. Atsushi Kato et al., Fagomine isomers and glycosides from Xanthocercis zambesiaca. Journal of Natural Products 1997, vol. 60, pp. 312-314
2. Hiroshi Nojima et al., “Antihyperglycemic effects of N-containing sugars from Xanthocercis zambesiaca, Morus bombycis, Aglaonema treubii, and Castanospermum australe in Streptozotocin-diabetic mice” Journal of Natural Product, 1998, vol 61. pp. 397-400.
3. Asano N. et al., Sugars with nitrogen in the ring isolated from the leaves of Morus bombycis, Carbohydrates Research 1994, vol. 254, pp 235-245.
4. Iqbal et al., Allelopathy of buckwheat: Assessment of allelopathic potential of extract of aerial parts of buckwheat and identification of fagomine and other related alkaloids as allelochemicals, Weed Biology and Management, 2002, vol. 2, pp. 110-115.
5. Kato et al., Fagomine isomers and glycosides from Xanthocersis zambesiaca, 1997, vol. 60, pp. 312-314.
6. United States patent application number US 20080014294.
7. European patent application number EP 949328.
8. Blaise P. Nic Phiarais, “Use of response surface methodology to investigate the effectiveness of commercial enzymes on buckwheat malt for brewing purposes”, Journal of the Institute of Brewing, 2006, vol 114(4), pp. 324-332.
9. United patent application number US20010018090.
10. Watson et al. “Polyhydroxylated alkaloids natural occurrence and therapeutic applications”, Phytochemistry, 2001, vol. 56, pp. 265-295.
11. Castillo et al. “Supporting information:Fructose-6-phosphate aldolase in organic synthesis: preparation of D-fagomine, N-alkylated derivatives and preliminary biological assays”, Internet citation, 29 Nov. 2006, pp. 51-S29, ISSN: 1523-7060.
12. Chen et al “Thermospray liquid chromatographic mass spectrometric analysis of castanospermine related alkaloids in castanospermun Australe”, Journal of natural products, 1990, vol. 53, n° 2, pp. 369-365.
13. “Practical methods for biocatalysis and biotransformation”, John Whittall et al, Ed. Wiley, page 216.
14. Sharon et al., “Bacterial adherence to cell surface sugars”, Ciba Found Symp., 1981, vol. 80, pp. 119-41.
15. Barira Islam et al., “Novel anti-adherence activity of mulberry leaves: Inhibition of Streptococcus mutans biofilm by 1-deoxynojirimycin isolated from Morus alba”, Journal of Antimicrobial Chemotherapy, 2008, vol. 62, pp. 751-757.
16. Ofek et al., “Bacterial adhesion to animal cells and tissues”, ASM Press, 2003, chapter 1.

1. A buckwheat extract comprising an amount of D-fagomine comprised between 2% and 40% by weight of dry extract mass and 3,4-di-epifagomine, wherein the weight ratio of 3,4-di-epifagomine/D-fagomine is comprised between 1:10 and 1:1, and the extract is substantially free of 1-deoxynojirimycin and 1,4 dideoxy-1,4-imino-D-arabinitol.

2. The extract according to claim 1, wherein the weight ratio of 3,4-di-epifagomine/D-fagomine is 1:2.

3. The extract according to claim 1, wherein the amount of D-fagomine is comprised between 5% and 18% by weight of dry extract mass.

4. The extract according to claim 1, wherein the amount of D-fagomine is comprised between 9% and 18% by weight of dry extract mass.

5. The extract according to claim 1, wherein the amount of D-fagomine is comprised between 12% and 18% by weight of dry extract mass.

6. The extract according to claim 1, wherein the amount of D-fagomine is 18% by weight of dry extract mass.

7. The extract according to claim 1, which is substantially free of fermentable sugars.

8. A process for the preparation of the extract as defined in claim 1, which comprises:
(a) milling the buckwheat, passing a sieve, and mixing it with water;
(b) mashing the mixture of step (a);
(c) carrying out an ethanolic fermentation of the extract obtained in step (b); (d) passing the fermented extract obtained in step (c) through a cation exchange resin, whereby the D-fagomine is retained;
(e) eluting the retained D-fagomine from the resin of step (d) with an alkaline buffer, and
(f) passing the extract obtained in step (e) through an anion exchange resin whereby D-fagomine is eluted directly.

9. The process according to claim 8, wherein the mashing step comprises the addition of exogenous enzymes.

10. The process according to claim 8, further comprising an additional step of passing the extract obtained in step (b) through an adsorption resin whereby the D-fagomine is eluted directly.

11. A functional food, dietary supplement, pharmaceutical or veterinary composition, which comprises the extract as defined in claim 1.

12. Use of the extract as defined in claim 1 as a blood glucose levels controlling agent to reduce post-prandial blood glucose levels after carbohydrate intake.

13. The extract as defined in claim 1, for use in the prevention and/or coadjuvant treatment of microbiota imbalance caused by an enteric or oral bacteria.

14. A process for the preparation of a substantially pure D-fagomine comprising carrying out the process as defined in claim 8, further comprising an additional step of passing the eluted fraction from step (f) through a high resolution cation exchange resin with terminal carboxymethyl groups whereby the D-fagomine is retained, and eluting the retained D-fagomine with an alkaline buffer.

15. The process according to claim 14 wherein the substantially pure D-fagomine is at least 90% by weight.

16. The process according to claim 14, wherein the substantially pure D-fagomine is at least 95% by weight.

17. The process according to claim 14, wherein the substantially pure D-fagomine is at least 99% by weight.
